(19) United States
(12) Patent Application Publication
(10) Pub. No.: US 2026/0207073 A1
(43) Pub. Date: Jul. 23, 2026

(54) ADAPTIVE DRIVING FREQUENCY ELECTRODERMAL ACTIVITY MEASUREMENT DEVICE AND METHODS

(71) Applicant: INNOPEAK TECHNOLOGY , INC., Palo Alto, CA (US)

(72) Inventor: Trevor HUDSON, Palo Alto, CA (US)

(73) Assignee: INNOPEAK TECHNOLOGY, INC., Palo Alto, CA (US)

(21) Appl. No.: 19/144,425

(22) PCT Filed: Dec. 29, 2022
(86) PCT No.: PCT/US2022/082526

§ 371 Date: Jun. 27, 2025

(51) Int. Cl. A61B 5/0531; A61B 5/00

(52) U.S. Cl. CPC A61B 5/0531; A61B 5/681

(57) ABSTRACT

A tuning circuit includes a first and second electrode disposed on a user, a processor for providing a first AC signal frequency to the electrodes, for measuring a first current from the electrodes, for providing a second AC signal frequency to the electrodes, for measuring a second current from the electrodes, for determining if the first current is greater than the second current, in response to the first current being greater than the second current, the processor is for providing the first AC signal frequency to the electrodes, for measuring a third current from the electrodes, and for determining a first electrodermal activity factor in response to the third current.

### CROSS REFERENCE TO RELATED APPLICATIONS

The present application is a 35 U.S.C. 371 National Phase application of International Patent Application No. PCT/US2022/082526, filed on Dec. 29, 2022, which is hereby incorporated by reference in its entirety.

### BACKGROUND

The present invention relates to smart wearable devices. More specifically, the present invention relates to improved methods for determining skin conductivity in a smart watch or other smart devices. Measuring the conductivity of the skin can be useful for a variety of applications, such as detecting changes in hydration levels, detecting the presence of certain chemicals on the skin, determining electrodermal activity, and the like. However, there are a number of challenges associated with using a smart device to measure skin conductivity.

One of the main challenges is that the skin's apparent conductivity can vary significantly depending on a number of factors, including humidity, temperature, the presence of sweat or other moisture, where the measurements are taken, and the like. Admittance-a characteristic related to conductivity and an inverse of impedance-changes due to various factors, and the changeability of admittance makes it difficult to measure conductivity with an AC source using constant measurement parameters.

This makes it difficult to accurately measure conductivity using a sensor on a smart device, as the readings may be influenced by these factors.

Another challenge is that the skin's apparent conductivity can vary significantly between different individuals, and even at different times of the day for a single individual. This makes it difficult to accurately capture and process conductivity readings between individuals, and even between subsequent measurements on the same individual.

Yet another challenge is that some solutions require an individual to stay relatively motionless during the test, for up to several minutes.

In light of the above, what is desired are methods and apparatus that can more accurately measure skin conductivity, with reduced drawbacks.

### SUMMARY OF THE PRESENT DISCLOSURE

The present invention relates to smart wearable devices.

According to one aspect, an electronic device is disclosed. One device may include a first electrode configured to contact a first portion of a user, and a second electrode configured to contact a second portion of the user. An apparatus may include a driving circuit coupled to the first electrode and the second electrode and configured to: determine a first condition indicating that a first stimulus signal is to be output between the first electrode and the second electrode, wherein in response to a determination, by the driving circuit, of the first condition, the driving circuit is caused to: determine a first primary frequency for the first stimulus signal, initiate outputting the first stimulus signal between the first electrode and the second electrode, determine a first feedback signal from the first electrode and the second electrode in response to the output of the first stimulus signal between the first electrode and the second electrode, and determine a second condition indicating that a second stimulus signal is to be output between the first electrode and the second electrode, and wherein in response to a determination, by the driving circuit, of the second condition, the driving circuit is caused to: determine a second primary frequency for the second stimulus signal, initiate output of the second stimulus signal between the first electrode and the second electrode, and determine a second feedback signal from the first electrode and the second electrode in response to the output of the second stimulus signal between the first electrode and the second electrode, wherein the first frequency of the first stimulus signal is different from the second frequency of the second stimulus signal. A system may include a processing circuit coupled to the driving circuit, wherein the processing circuit is configured to: specify the first condition to the driving circuit for a first period of time, receive the first feedback signal from the driving circuit, determine a first magnitude of the first feedback signal, specify the second condition to the driving circuit for a second period of time, receive the second feedback signal from the driving circuit, determine a second magnitude of the second feedback signal, determine whether the first magnitude exceeds the second magnitude, wherein in response to a determination by the processing circuit, that the first magnitude exceeds the second magnitude, the processing circuit is caused to: specify the first condition to the driving circuit for a third period of time.

According to another aspect, a method for an electronic device having a first electrode contacting a first portion of a user and a second electrode contacting a second portion of the user is disclosed. One technique includes specifying, with a processing circuit, a first condition to a driving circuit for a first period of time, determining, with the driving circuit, the first condition indicating that a first stimulus signal is to be output between the first electrode and the second electrode, wherein in response to determining, by the driving circuit, the first condition, the method includes determining, with the driving circuit, a first primary frequency for the first stimulus signal, initiating output, with the driving circuit, if the first stimulus signal between the first electrode and the second electrode, determining, with the driving circuit, a first feedback signal from the first electrode and the second electrode in response to the output of the first stimulus signal between the first electrode and the second electrode, receiving, with the processing circuit, the first feedback signal from the driving circuit, and determining, with the processing circuit, a first magnitude of the first feedback signal. A process may include specifying, with the processing circuit, a second condition to the driving circuit for a second period of time, determining, with the driving circuit, the second condition indicating that a second stimulus signal is to be output between the first electrode and the second electrode, wherein in response to determining, by the driving circuit, the second condition, the method further includes: determining, with the driving circuit, a second primary frequency for the second stimulus signal, initiating output, with the driving circuit, of the second stimulus signal between the first electrode and the second electrode, determining, with the driving circuit, a second feedback signal from the first electrode and the second electrode in response to the output of the second stimulus signal between the first electrode and the second electrode, wherein the first frequency of the first stimulus signal is different from the second frequency of the second stimulus signal, receiving, with the processing circuit, the second feedback signal from the driving circuit, and determining, with the processing circuit, a second magnitude of the second feedback signal. A process may include determining, with the processing circuit, whether the first magnitude exceeds the second magnitude, and specifying, with the processing circuit, the first condition to the driving circuit for a third period of time, in response to determining that the first magnitude exceeds the second magnitude.

According to yet another aspect, a tuning circuit is disclosed. One device may include a first electrode configured to be disposed against a first location on a test surface, and a second electrode configured to be disposed against a second location on the test surface. An apparatus may include a processing module electrically coupled to the first electrode and the second electrode, wherein the processing module is configured to provide a first AC voltage signal characterized by a first frequency across the first electrode and the second electrode, wherein the processing module is configured to measure a first current flowing between the first electrode and the second electrode in response to the first AC voltage signal, wherein the processing module is configured to determine a first responsive signal in response to the first current and to the first AC voltage signal, wherein the processing module is configured to provide a second AC voltage signal characterized by a second frequency across the first electrode and the second electrode, wherein the second frequency is different from the first frequency, and wherein the processing module is configured to measure a second current flowing between the first electrode and the second electrode in response to the second AC voltage signal, wherein the processing module is configured to determine a second responsive signal in response to the second current and to the second AC voltage signal. A system may include a processing module configured to determine if the first responsive signal is greater than the second responsive signal, wherein in response to determining if the first responsive signal is greater than the second responsive signal, the processing module is configured to provide a third AC voltage signal characterized by the first frequency across the first electrode and the second electrode, wherein the processing module is configured to measure a third current flowing between the first electrode and the second electrode in response to the third AC voltage signal, and wherein the processing module is configured to determine a first electrodermal activity factor in response to the third current and to the third AC voltage signal.

### BRIEF DESCRIPTION OF THE DRAWINGS

In order to more fully understand the present invention, reference is made to the accompanying drawings. Understanding that these drawings are not to be considered limitations in the scope of the invention, the presently described embodiments and the presently understood best mode of the invention are described with additional detail through use of the accompanying drawings in which:

FIG. 1 illustrates a functional block diagram of some embodiments of the present invention;

FIG. 2 illustrates a process diagram according to various embodiments of the present invention; and

FIG. 3 illustrates another process diagram according to various embodiments of the present invention.

### DETAILED DESCRIPTION

The present invention relates to methods and apparatus for measuring skin conductivity or the like. More specifically, the present invention relates to methods and apparatus for measuring changes of resistance in the epidermis due to sweat, or the like. Initially, embodiments apply an alternating current (AC) stimulus signal to electrodes placed against the user's skin. The primary frequency of the stimulus signal can then be varied or tuned to increase the resulting measurements.

In some embodiments, an electrical impedance of skin may be modeled as an electrode-skin interface in series with a body impedance. The electrode-skin interface may be modeled as a capacitance in parallel with a resistor. The body impedance may be modeled as a capacitance and resistor (together, epidermis impedance) in parallel with one or more variable resistances (the resistance of interest herein). Typically the latter resistances may vary depending upon the amount of moisture (e.g. sweat) within the skin.

Using AC signals to determine skin conductivity may provide several benefits. As discussed above, the electrical impedance of an electrode-skin interface can be represented as a resistor and capacitor in parallel and be expressed mathematically as shown below. Here, j is the imaginary unit and @ is angular frequency.

$${Z}_{interface} = \frac{1}{j ω C + 1 / R}$$

In various embodiments, higher frequencies may result in a lower electrical impedance of the interface. A lower impedance may improve the signal-to-noise ratio (SNR) of the desired measurement, because the total current magnitude through the body increases at higher frequencies.

However, with respect to the body impedance, in various embodiments, at higher frequencies, more current may be shunted through the epidermis impedance, rather than the variable resistance elements (e.g. activated sweat ducts). This leads to a lower SNR because less current is passing through the variable resistances of interest. Additionally, in various embodiments, higher frequencies may be beneficial for measuring partially filled sweat ducts, as the signal can better penetrate through the stratum corneum of the skin to reach a submerged fluid front of the sweat duct.

In some cases, the skin characteristics of an individual may be static, for example, the number of cell layers, tissue composition, skin thickness, sweat gland density and duct dimensions, hair density and thickness, degree of electrode corrosion or oxidation, and other factors which affect the equivalent circuit. Additionally, the skin characteristics may be dynamic, such as the height of electrolytes in the sweat gland ducts, hydration level of skin tissue, amount of skin surface wetting, degree of electrode-skin contact, and the like. These factors can change the frequency response of the skin circuit.

In light of the above, an optimal frequency for measurement of the skin conductivity may vary depending on individual variation and may involve a local maximum or the like in SNR along the frequency spectrum.

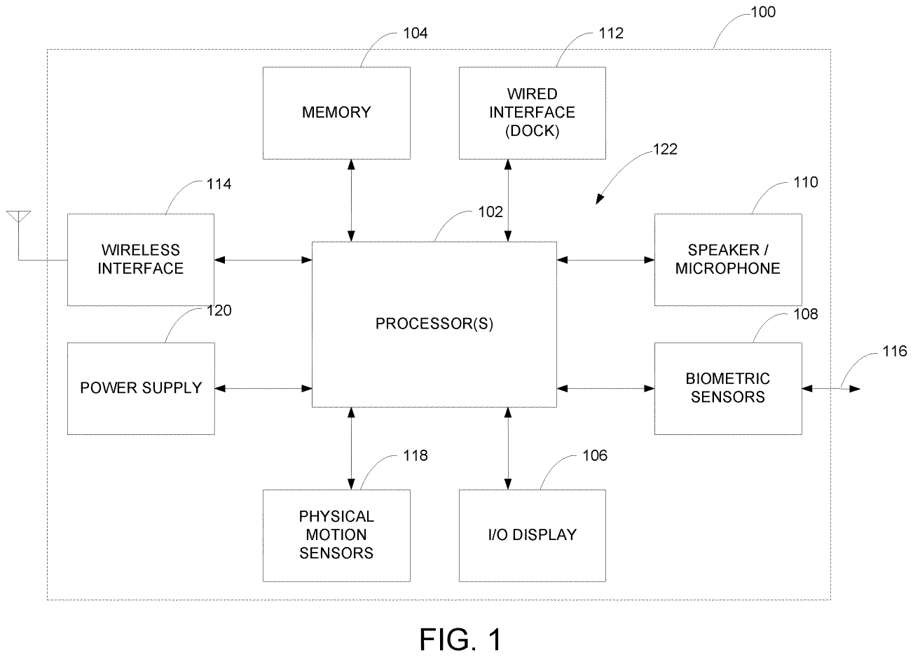

FIG. 1 illustrates a functional block diagram of some embodiments of the present invention. It is contemplated that embodiments such as smartwatches, fitness trackers, augmented reality (AR) devices, smart headphones, and the like may be implemented with a subset or superset of the below illustrated components. In FIG. 1, a computing device 100 typically includes an applications processor 102, memory 104, a display 106, biometric sensors 108, audio input/output devices 110, and the like. One or more buses 122 may be used to provide intercommunication between the functional blocks illustrated in FIG. 1.

Communications between computing device 100 and other devices may include a wired interface 112 or a wireless interface 114. In some embodiments, the wired interface 112 may include a universal serial bus (e.g. USB), high definition multimedia interface (HDMI), serial communications, custom interface and associated docking interface, or the like. In other embodiments, interface 114 may utilize physical contact or close physical contact of device 100 to a dock for the transfer of data, magnetic power, heat energy, electrical power or the like. In additional embodiments, no wired interface 112 may be included.

In some embodiments, wireless interface 114 may include cellular communication such as: long-term evolution (LTE), long-term evolution-advanced (LTE-A), code division multiple access (CDMA), wideband code division multiple access (WCDMA), universal mobile telecommunication system (UMTS), wireless broadband (WiBro), global system for mobile communication (GSM), G4, G5, or other wireless network technology. Other types of wireless communication that may be supported may include: wireless-fidelity (Wi-Fi), light-fidelity (Li-Fi), Bluetooth, Bluetooth low power (BLE), ZigBee, near-field communication (NFC), magnetic secure transmission (MST), radio frequency (RF), ultra-wide-band (UWB), infrared (IR), mesh-network, or the like. In some embodiments, wireless interface 114 may include global navigation satellite system (GNSS). The GNSS may be, e.g., global positioning system (GPS), global navigation satellite system (Glonass), Beidou navigation satellite system (hereinafter, “Beidou”) or Galileo, or the European global satellite-based navigation system. Hereinafter, the terms “GPS” and the “GNSS” may be interchangeably used herein.

In some embodiments, a number of sensors are provided including biometric sensors 108 for capturing biological data via interface 116 associated with a user or wearer, and motion sensors 118 for capturing movement data of the user or wearer. In some embodiments, motion sensors 118 may include microelectronic and micromechanical (MEMS) devices such as accelerometers, gyroscopes, magnetometers, pressure sensors and the like. For sake of convenience, haptic output devices are grouped within physical motion sensors 118. These haptic devices may be provided to provide physical vibrations as feedback to a wearer of computing device 100.

Biometric sensors 108 and interface 116 may include light sources and sensors, such as LEDs that emit infrared, visible light or the like, and light sensors that receive light reflected from a user. These sources and sensors may be used to monitor blood oxygen levels, pulse rates, blood sugar levels, and the like; and to capture images of fingerprints, blood vessel patterns, and the like. In various embodiments, biometric sensors 108 may also include embodiments of the present invention. As will be described below, embodiments may include an electrical stimulus driver circuit that outputs electrical stimulus signals, an electrical receiver circuit that receives responsive biological signals, and interface 116 may include electrodes that impart the electrical stimulus signals to the user or wearer and receives the response biological signals from the wearer. In some embodiments, some of the functionality described below may be proportioned in different ways between the processor and driver circuit.

In various embodiments, computing device 100 may include one or more processors 102. Such processors 102 may also be termed application processors, and may include a processor core, a video/graphics core, and other functional cores illustrated in FIG. 1. Processors 102 may include processors from Apple (e.g. S7, S8), Qualcomm (e.g. Snapdragon W5, W5+), Samsung (e.g. Exynos 1280, 2200), ARM (e.g. ARMv7-A), and the like. In some embodiments, processing accelerators may also be included, e.g. an AI accelerator, Google (Tensor processing unit (TPU), a GPU, or the like. It is contemplated that other existing and/or later-developed processors may be used in various embodiments of the present invention.

In various embodiments, memory 104 may include different types of memory (including memory controllers), such as flash memory (e.g. NOR, NAND), SRAM, DDR SDRAM, or the like. Memory 104 may be fixed within computing device 100 and may include removable (e.g. MICRO SD). The above are examples of computer-readable tangible media that may be used to store embodiments of the present invention, such as computer-executable software code (e.g. firmware, application programs), application data (e.g. biometric data, application data, secure element data, operating system data, or the like. It is contemplated that other existing and/or later-developed memory and memory technology may be used in various embodiments of the present invention.

In various embodiments, display 106 may be based upon a variety of later-developed or current display technology. In some examples, display 106 may include, e.g., a liquid crystal display (LCD), a light emitting diode (LED) display, an organic light emitting diode (OLED) display, or a microelectromechanical systems (MEMS) display, or an electronic paper display, or the like, but is not limited thereto. Additionally display 106 may display, e.g., various contents (e.g., text, images, videos, icons, or symbols) to the user. Display 160 may also include a touchscreen and may receive input from a user, e.g., a touch, gesture, proximity, or hovering input using an electronic pen or a body portion of the user. In various embodiments, the resolution of such displays and the resolution of such touch sensors may be set based upon engineering or non-engineering factors (e.g. sales, marketing).

In some embodiments, audio input/output 110 may include microphone(s)/speakers. In various embodiments, voice processing and/or recognition software may be provided to applications processor 102 to enable the user to operate computing device 100 by stating voice commands. In various embodiments of the present invention, audio input 110 may provide user input data in the form of a spoken word or phrase, or the like. Audio output 110 may provide sound responsive to user input via the touchscreen of display 106, or other user input (e.g. buttons, dials, etc.).

In various embodiments, any number of future developed, current operating systems, or custom operating systems may be supported, such as Apple WatchOS, Google Wear OS, Samsung Tizen OS, Fitbit OS, Garmin Watch OS, and the like. In various embodiments of the present invention, the operating system may be a multi-threaded multi-tasking operating system. Accordingly, inputs and/or outputs from and to display 106 and inputs and/or outputs to biometric sensors 108 may be processed in parallel processing threads. Inputs and outputs from other functional blocks may also be processed in parallel or serially, in other embodiments.

In some embodiments, a power supply 120, e.g. battery (e.g. LiPo), ultracapacitor, or the like, is included that provides operating electrical power to device 100. In various embodiments, any number of power generation techniques may be utilized to supplement or even replace power supply 120, such as solar power, liquid metal power generation, thermoelectric engines, or the like.

FIG. 1 is representative of one computing device 100 capable of embodying the present invention. It will be readily apparent to one of ordinary skill in the art that many other hardware and software configurations are suitable for use with the present invention. Embodiments of the present invention may include at least some but need not include all of the functional blocks illustrated in FIG. 1.

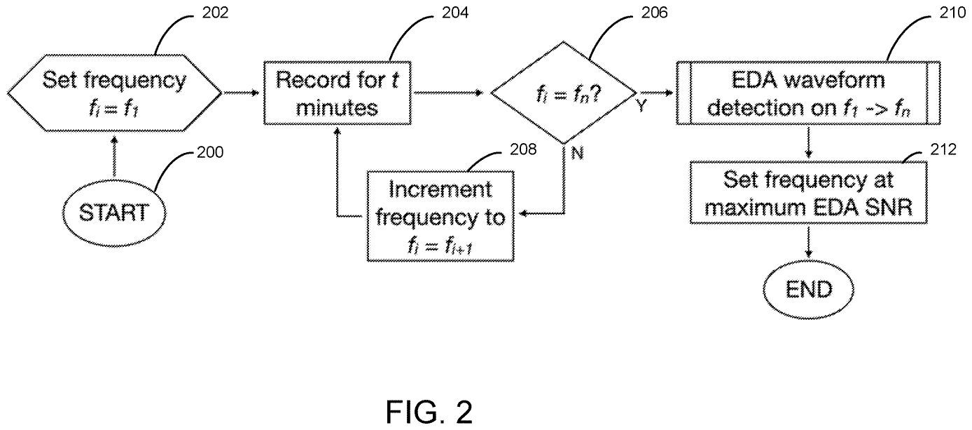

FIG. 2 illustrates a block diagram of a process according to various embodiments. More specifically, a method is illustrated to adjust a driving frequency of a measurement circuit programmatically to adapt to the factors listed above.

Initially, a smart device (e.g. a smartwatch, a smart ring, a fitness tracker) is put on by a user, step 200. Next, a processor in the smartwatch (or a driving circuit) may determine a first frequency, step 202. In some examples, the first frequency may be a default frequency (e.g. 20 Hz) selected within a range of 1 Hertz to 100 Hertz, or the like. Alternatively, the first frequency may be the same frequency used in during a previous period of time the smart device is worn by the user and stored in memory.

In various embodiments, a driving circuit may generate a first stimulus signal with the first frequency. The driving circuit outputs the first stimulus signal via electrodes (e.g. first and second electrodes) that contact the user's skin. For example, if a wrist-worn device, the electrodes may be positioned on the back-side of the device touching the user's wrist, or the like.

In various embodiments, in response to the first stimulus signal, first skin conductivity data, in the form of current conduction may be measured between the electrodes. In FIG. 2, this first measurement may be performed by a specific period of time, e.g. seconds, minutes, or the like, step 204. Subsequently, the processor or driving circuit may determine whether to adjust the applied frequency or not, step 206.

In various embodiments, the processor or the driving circuit may determine a second frequency, step 208. This second frequency is typically different from the first applied frequency, and may represent an incremental or decremental frequency. In some examples, the second frequency may be a default frequency (e.g. 50 Hz) or selected within a range of 1 Hertz to 100 Hertz, or the like. In one example, the first frequency may be 10 Hertz and the second frequency may be 15 Hertz or the like.

As illustrated, steps 204 and 206 may be repeated using the second frequency. Step 208 may then be repeated for a third or a fourth frequency. As an example, the first frequency may be 30 Hz, the second frequency may be 40 Hz, the third frequency may be 20 Hz, and the like. As mentioned above, these data may be recorded and associated with the respective, applied frequencies. The number of iterations may range from one to ten, two to twenty, and the like. In some cases, the number of frequencies may range from twenty to fifty, or more.

In various embodiments, the measurements may be processed in the processor to determine skin conductance data or other computed values, step 210. These values are then compared to each other to determine the desired skin conductance value (and desired stimulus frequency). In some examples, the skin conductance that has the highest magnitude is selected, the skin conductance with the highest SNR magnitude (e.g., of the baseline conductivity level) is selected, or the like. In response to the selected skin conductance, the selected stimulus frequency associated with that skin conductance is determined, step 212.

In various embodiments, an electrodermal activity waveform detection algorithm is used by the processor to determine the stimulus frequency with a large or largest SNR (e.g., of the peak heigh, or the deflection of conductivity away from the baseline). In some embodiments, the algorithm may also identify multiple peaks recorded, in order to return a peak amplitude standard deviation. These standard deviations could also be used along with the SNR data to make inferences about the current frequency. For example, even though a frequency is associated with the highest SNR, if it has a standard deviation above a threshold, that frequency may be disqualified. Accordingly, other parameters than simply the SNR may be used to determine the appropriate stimulus frequency for this session.

In various embodiments, the selected stimulus frequency is then used as a default stimulus frequency for measuring skin conductance during this user session. In some embodiments, this session may be until the user takes off the wearable device. In other embodiments, this session may be for a limited period of time, for example, an hour, a day, a week, or the like. After the period of time, the process discussed above may again be performed to determine a stimulus frequency that is appropriate for the then current user skin conditions. In other embodiments, the session may be based upon other factors, for example, after a certain amount of activity (e.g. calories, active hours, etc.) is detected, after a workout is completed, and the like. After these events, the process discussed above may again be performed.

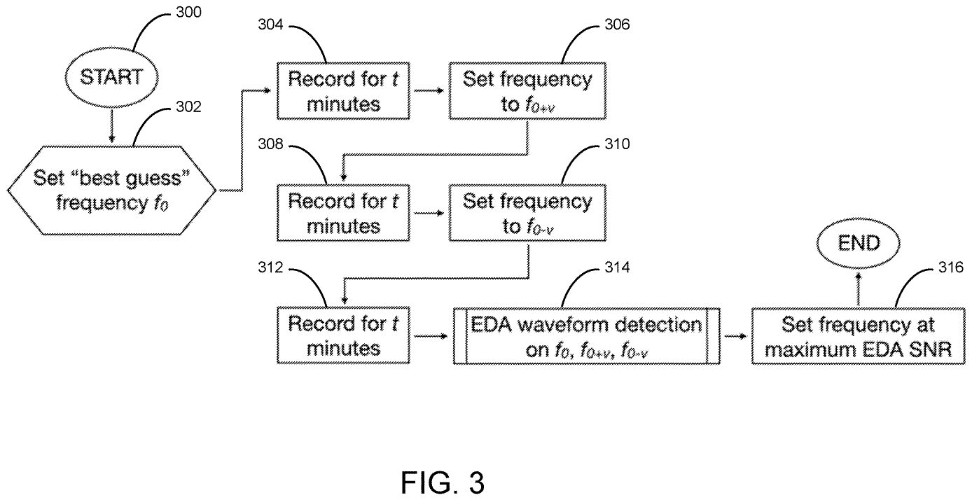

FIG. 3 illustrates another block diagram of another process according to various embodiments. More specifically, a method is illustrated to adjust a driving frequency of a measurement circuit programmatically to adapt to the factors listed above.

Initially, a smart device is worn by a user, step 300. Additionally, a processor may specify a default frequency or a first selected frequency associated with a desired SNR, described in FIG. 2 above, step 302. Similar to above, in some embodiments the frequency is typically within a range of 1 Hertz to 100 Hertz, 100 Hertz to 250 Hertz, or the like.

In various embodiments, in response to the first frequency, or the like, a driving circuit may generate a first stimulus signal with the first frequency. The driving circuit outputs the first stimulus signal via electrodes (e.g. first and second electrodes) that contact the user's skin, step 304. In response to the first stimulus signal, skin conductivity data, e.g. a first current, or the like may be measured between the electrodes. In FIG. 3, this measurement may be performed over a specific period of time, e.g. seconds, minutes, or the like. The first current may be stored or buffered in memory.

In various embodiments, the processor or the driving circuit may then determine a second frequency, step 306. This second frequency is typically different from the first frequency, and may represent a frequency increment. In some examples, the second frequency may also be within a range of 1 Hertz to 100 Hertz, or the like. In one example, the first frequency may be 50 Hertz and the second frequency may be 65 Hertz or the like.

In various embodiments, in response to the second frequency, a driving circuit may generate a second stimulus signal (with the second frequency). The driving circuit outputs the second stimulus signal via electrodes (e.g. first and second electrodes) that contact the user's skin, step 308. In response to the second stimulus signal, skin conductivity data, a second current, or the like may be measured between the electrodes. In FIG. 3, this measurement may be performed over a specific period of time, e.g. seconds, minutes, or the like. The duration may be similar or different from the duration specified in step 304, above. The second current may be stored or buffered in memory.

In various embodiments, the processor or the driving circuit may then determine a third frequency, step 310. This third frequency is typically different from the first or second frequencies, and may represent a frequency decrement. In some examples, the third selected frequency may also be within a range of 1 Hertz to 100 Hertz, or the like. In one example, the first selected frequency may be 75 Hertz, a second selected frequency may be 100 Hertz, and the third selected frequency is 40 Hertz or the like.

In various embodiments, in response to the third frequency, or the like, a driving circuit may generate a third stimulus signal with the third frequency. The driving circuit outputs the third stimulus signal via electrodes (e.g. first and second electrodes) that contact the user's skin, step 312. In response to third stimulus signal, skin conductivity data, a third current, or the like may be measured between the electrodes. In FIG. 3, this measurement may be performed again over a specific period of time, e.g. seconds, minutes, or the like. The duration may be similar or different from the duration specified in steps 304 and 308 above. The third current may also be stored or buffered in memory.

In some embodiments, this process may be repeated any number of times, e.g. 10, 20, 50 times to result in the same number of recorded current profiles. The number of iterations is typically limited by the amount of power consumed for each iteration. The greater the number of iterations, the greater the power draw and the shorter the operation time for the smart device.

Subsequently, the recorded current measurements may be processed in the processor to determine skin conductance data or other computed values (e.g. electrodermal activity), step 314. These computed values are then compared to each other. In some examples, the skin conductance or electrodermal activity that has the highest magnitude, with the highest SNR magnitude is selected, and the like. In other examples, the computed data that has a high magnitude and a low standard deviation is selected and the associated stimulus frequency is determined.

In various embodiments, the newly selected stimulus frequency is then used as a default stimulus frequency for measuring skin conductance during this user session, step 316. Similar to above, this session may last: until the user takes off the wearable device; for a limited period of time, for example, an hour, a day, a week, or the like; be based upon other factors, for example, after a certain amount of activity (e.g. calories, active hours, etc.) is detected, after a workout is completed; and the like. Subsequently, the process discussed above may be performed again upon the occurrence of another event.

The technical solutions of the present disclosure may be applied in the field of EDA data collection.

Some embodiments of the present disclosure may provide an electronic device. The electronic device may include a first electrode configured to contact a first portion of a user, a second electrode configured to contact a second portion of the user, and a driving circuit coupled to the first electrode and the second electrode. The driving circuit may be configured to: determine a first condition indicating that a first stimulus signal is to be output between the first electrode and the second electrode. In response to a determination, by the driving circuit, of the first condition, the driving circuit may be caused to: determine a first primary frequency for the first stimulus signal; initiate outputting the first stimulus signal between the first electrode and the second electrode; determine a first feedback signal from the first electrode and the second electrode in response to the output of the first stimulus signal between the first electrode and the second electrode; and determine a second condition indicating that a second stimulus signal is to be output between the first electrode and the second electrode. In response to a determination, by the driving circuit, of the second condition, the driving circuit may be caused to: determine a second primary frequency for the second stimulus signal; initiate output of the second stimulus signal between the first electrode and the second electrode; and determine a second feedback signal from the first electrode and the second electrode in response to the output of the second stimulus signal between the first electrode and the second electrode. The first frequency of the first stimulus signal may be different from the second frequency of the second stimulus signal.

In some embodiments, the driving circuit may be also caused to: repeat to determine a second primary frequency for the second stimulus signal by an increment frequency or a decrement frequency; initiate output of the second stimulus signal between the first electrode and the second electrode; and determine a second feedback signal from the first electrode and the second electrode in response to the output of the second stimulus signal between the first electrode and the second electrode.

In some embodiments, the electronic device may be applied in EDA data collection and further include a processing circuit coupled to the driving circuit. The processing circuit may be configured to: specify the first condition to the driving circuit for a first period of time; receive the first feedback signal from the driving circuit; determine a first magnitude of the first feedback signal; specify the second condition to the driving circuit for a second period of time; receive the second feedback signal from the driving circuit; determine a second magnitude of the second feedback signal.

In some embodiments, the processing circuit may be also configured to: determine whether the first magnitude exceeds the second magnitude. In response to a determination by the processing circuit, that the first magnitude exceeds the second magnitude, the processing circuit may be caused to: specify the first condition to the driving circuit for a third period of time. In some embodiments, the processing circuit may be also configured to: determine whether the second magnitude exceeds the first magnitude. In response to a determination by the processing circuit, that the second magnitude exceeds the first magnitude, the processing circuit may be caused to: specify the second condition to the driving circuit for a fourth period of time.

In some embodiments, in response to a determination, by the driving circuit, of a third condition, the driving circuit may be caused to: determine a third primary frequency for a third stimulus signal; initiate output of the third stimulus signal between the first electrode and the second electrode; and determine a third feedback signal from the first electrode and the second electrode in response to the output of the third stimulus signal between the first electrode and the second electrode. The third frequency of the third stimulus signal may be different from the second frequency of the second stimulus signal and the first frequency of the first stimulus signal.

In some embodiments, the first frequency may be a default frequency or a selected frequency associated with a desired SNR. The second frequency may be a frequency by adding the first frequency with an increment frequency. The third frequency may be a frequency by subtracting the first frequency with a decrement frequency.

In some embodiments, the processing circuit may be configured to: specify the first condition to the driving circuit for a first period of time; receive the first feedback signal from the driving circuit; determine a first magnitude of the first feedback signal; specify the second condition to the driving circuit for a second period of time; receive the second feedback signal from the driving circuit; determine a second magnitude of the second feedback signal; specify the third condition to the driving circuit for a third period of time; receive the third feedback signal from the driving circuit; determine a third magnitude of the third feedback signal.

In some embodiments, the processing circuit may be also configured to: determine a highest magnitude among the first magnitude, the second magnitude, and the third magnitude. In response to a determination by the processing circuit, that the first magnitude is the highest magnitude, the processing circuit may be caused to: specify the first condition to the driving circuit for the third period of time. In response to a determination by the processing circuit, that the second magnitude is the highest magnitude, the processing circuit may be caused to: specify the second condition to the driving circuit for the fourth period of time. In response to a determination by the processing circuit, that the third magnitude is the highest magnitude, the processing circuit may be caused to: specify the third condition to the driving circuit for a fifth period of time.

In some embodiments, the first magnitude of the first feedback signal may be selected from a group consisting of: a signal to noise ratio, and a maximum magnitude.

In some embodiments, the first period of time and the second period of time may be substantially similar in duration. The first period of time may be smaller than the third period of time.

Some embodiments of the present disclosure may further provide a method for an electronic device. The electronic device may have a first electrode contacting a first portion of a user and a second electrode contacting a second portion of the user. The method may include: determining, with the driving circuit, a first condition indicating that a first stimulus signal is to be output between the first electrode and the second electrode. In response to determining, by the driving circuit, the first condition, the method further may include: determining, with the driving circuit, a first primary frequency for the first stimulus signal; initiating output, with the driving circuit, of the first stimulus signal between the first electrode and the second electrode; and determining, with the driving circuit, a first feedback signal from the first electrode and the second electrode in response to the output of the first stimulus signal between the first electrode and the second electrode; determining, with the driving circuit, a second condition indicating that a second stimulus signal is to be output between the first electrode and the second electrode. In response to determining, by the driving circuit, the second condition, the method further may include: determining, with the driving circuit, a second primary frequency for the second stimulus signal; initiating output, with the driving circuit, if the second stimulus signal between the first electrode and the second electrode; and determining, with the driving circuit, a second feedback signal from the first electrode and the second electrode in response to the output of the second stimulus signal between the first electrode and the second electrode. The first frequency of the first stimulus signal may be different from the second frequency of the second stimulus signal.

In some embodiments, the method may further include: in response to a determination, by the driving circuit, of a third condition, determine, with the driving circuit, a third primary frequency for a third stimulus signal; initiate, with the driving circuit, output of the third stimulus signal between the first electrode and the second electrode; and determine, with the driving circuit, a third feedback signal from the first electrode and the second electrode in response to the output of the third stimulus signal between the first electrode and the second electrode. The third frequency of the third stimulus signal may be different from the second frequency of the second stimulus signal and the first frequency of the first stimulus signal.

In some embodiments, the method may further include: specifying, with a processing circuit, a first condition to a driving circuit for a first period of time; receiving, with the processing circuit, the first feedback signal from the driving circuit; determining, with the processing circuit, a first magnitude of the first feedback signal; specifying, with the processing circuit, a second condition to the driving circuit for a second period of time; receiving, with the processing circuit, the second feedback signal from the driving circuit; determining, with the processing circuit, a second magnitude of the second feedback signal; determine, with the processing circuit, whether the first magnitude exceeds the second magnitude; in response to a determination by the processing circuit, that the first magnitude exceeds the second magnitude, specify, with the processing circuit, the first condition to the driving circuit for a third period of time; or determine, with the processing circuit, whether the second magnitude exceeds the first magnitude; in response to a determination by the processing circuit, that the second magnitude exceeds the first magnitude, specify, with the processing circuit, the second condition to the driving circuit for a fourth period of time; specify, with the processing circuit, the first condition to the driving circuit for a first period of time; receive, with the processing circuit, the first feedback signal from the driving circuit; determine, with the processing circuit, a first magnitude of the first feedback signal; specify, with the processing circuit, the second condition to the driving circuit for a second period of time; receive, with the processing circuit, the second feedback signal from the driving circuit; determine, with the processing circuit, a second magnitude of the second feedback signal; specify, with the processing circuit, the third condition to the driving circuit for a third period of time; receive, with the processing circuit, the third feedback signal from the driving circuit; determine, with the processing circuit, a third magnitude of the third feedback signal; determine, with the processing circuit, a highest magnitude among the first magnitude, the second magnitude, and the third magnitude; in response to a determination by the processing circuit, that the first magnitude is the highest magnitude, specify, with the processing circuit, the first condition to the driving circuit for the third period of time; in response to a determination by the processing circuit, that the second magnitude is the highest magnitude, specify, with the processing circuit, the second condition to the driving circuit for the fourth period of time; in response to a determination by the processing circuit, that the third magnitude is the highest magnitude, specify, with the processing circuit, the third condition to the driving circuit for a fifth period of time.

In some embodiments, the first magnitude of the first feedback signal may be selected from a group consisting of: a signal to noise ratio, and a maximum magnitude.

In some embodiments, the first period of time and the second period of time may be substantially similar in duration. The first period of time may be smaller than the third period of time.

Some embodiments of the present disclosure may further provide a tuning circuit. The tuning circuit may include first electrode configured to be disposed against a first location on a test surface, a second electrode configured to be disposed against a second location on the test surface, and a processing module electrically coupled to the first electrode and the second electrode. The processing module may be configured to provide a first AC voltage signal characterized by a first frequency across the first electrode and the second electrode. The processing module may be configured to measure a first current flowing between the first electrode and the second electrode in response to the first AC voltage signal. The processing module may be configured to determine a first responsive signal in response to the first current and to the first AC voltage signal. The processing module may be configured to provide a second AC voltage signal characterized by a second frequency across the first electrode and the second electrode. The second frequency may be different from the first frequency. The processing module may be configured to measure a second current flowing between the first electrode and the second electrode in response to the second AC voltage signal. The processing module may be configured to determine a second responsive signal in response to the second current and to the second AC voltage signal. Thereafter the processing module may be configured to determine if the first responsive signal is greater than the second responsive signal. In response to determining if the first responsive signal is greater than the second responsive signal, the processing module may be configured to provide a third AC voltage signal characterized by the first frequency across the first electrode and the second electrode. The processing module may be configured to measure a third current flowing between the first electrode and the second electrode in response to the third AC voltage signal. The processing module may be configured to determine a first electrodermal activity factor in response to the third current and to the third AC voltage signal.

In some embodiments, a first magnitude of the first AC voltage signal, a second magnitude of the second AC voltage signal, and a third magnitude of the third AC voltage signal may be substantially similar.

In some embodiments, in response to determining if the first responsive signal is not greater than the second responsive signal: the processing module may be configured to provide a fourth AC voltage signal characterized by the second frequency across the first electrode and the second electrode. The processing module may be configured to measure a fourth current flowing between the first electrode and the second electrode in response to the fourth AC voltage signal. The processing module may be configured to determine a second electrodermal activity factor in response to the fourth current and to the fourth AC voltage signal.

In some embodiments, the tuning circuit may be configured in an electronic device that is applied in EDA data collection. The test surface may be selected from a group consisting of: a wrist, a finger, fingers, a palm, a foot and a portion of a body.

In some embodiments, the processing module may be configured to provide the first AC voltage signal characterized by the first frequency across the first electrode and the second electrode in response to an event. The event may be selected from a group consisting of: elapse of an amount of time, when a magnitude of the third current flowing between the first electrode and the second electrode is less than a predetermined magnitude, upon initialization of the tuning circuit, at a predetermined time of day.

In light of the above, other variations and adaptations can be envisioned to one of ordinary skill in the art. For example, the number of events that can trigger a redetermination of a stimulus frequency can include more than one of the ones described above, including, whether the user is asleep or awake, the weather conditions, and the like. Additionally, some types of events may delay when the stimulus frequency is determined, such as when the user is performing high-impact activities (e.g. boxing, driving), when the user is showering or when the smart device is put in “water” mode, and the like. Additionally, in some embodiments, the smart device may be a smartwatch, a fitness tracker, a smart ring, smart glasses, an augmented reality device, earbuds, and the like.

The block diagrams of the architecture and flow charts are grouped for ease of understanding. However, it should be understood that combinations of blocks, additions of new blocks, re-arrangement of blocks, and the like are contemplated in alternative embodiments of the present invention. The specification and drawings are, accordingly, to be regarded in an illustrative rather than a restrictive sense. It will, however, be evident that various modifications and changes may be made thereunto without departing from the broader spirit and scope of the invention as set forth in the claims.

1-20. (canceled)

21. An electronic device, comprising:
a first electrode configured to contact a first portion of a user;
a second electrode configured to contact a second portion of the user;
a driving circuit coupled to the first electrode and the second electrode and configured to:
determine a first condition indicating that a first stimulus signal is to be output between the first electrode and the second electrode;
wherein in response to a determination, by the driving circuit, of the first condition, the driving circuit is caused to:
determine a first primary frequency for the first stimulus signal;
initiate outputting the first stimulus signal between the first electrode and the second electrode;
determine a first feedback signal from the first electrode and the second electrode in response to the output of the first stimulus signal between the first electrode and the second electrode; and
determine a second condition indicating that a second stimulus signal is to be output between the first electrode and the second electrode;
wherein in response to a determination, by the driving circuit, of the second condition, the driving circuit is caused to:
determine a second primary frequency for the second stimulus signal;
initiate output of the second stimulus signal between the first electrode and the second electrode; and
determine a second feedback signal from the first electrode and the second electrode in response to the output of the second stimulus signal between the first electrode and the second electrode; and
wherein the first frequency of the first stimulus signal is different from the second frequency of the second stimulus signal.

22. The electronic device of claim 21, wherein the driving circuit is also caused to:
repeat to determine a second primary frequency for the second stimulus signal by an increment frequency or a decrement frequency; initiate output of the second stimulus signal between the first electrode and the second electrode; and determine a second feedback signal from the first electrode and the second electrode in response to the output of the second stimulus signal between the first electrode and the second electrode.

23. The electronic device of claim 21, the electronic device applied in electrodermal activity (EDA) data collection and, further comprising a processing circuit coupled to the driving circuit and the processing circuit is configured to:
specify the first condition to the driving circuit for a first period of time;
receive the first feedback signal from the driving circuit;
determine a first magnitude of the first feedback signal;
specify the second condition to the driving circuit for a second period of time;
receive the second feedback signal from the driving circuit;
determine a second magnitude of the second feedback signal.

24. The electronic device of claim 23, wherein the processing circuit is also configured to:
determine whether the first magnitude exceeds the second magnitude;
wherein in response to a determination by the processing circuit, that the first magnitude exceeds the second magnitude, the processing circuit is caused to:
specify the first condition to the driving circuit for a third period of time; or
determine whether the second magnitude exceeds the first magnitude;
wherein in response to a determination by the processing circuit, that the second magnitude exceeds the first magnitude, the processing circuit is caused to:
specify the second condition to the driving circuit for a fourth period of time.

25. The electronic device of claim 21, wherein in response to a determination, by the driving circuit, of a third condition, the driving circuit is caused to:
determine a third primary frequency for a third stimulus signal;
initiate output of the third stimulus signal between the first electrode and the second electrode; and
determine a third feedback signal from the first electrode and the second electrode in response to the output of the third stimulus signal between the first electrode and the second electrode;
wherein the third frequency of the third stimulus signal is different from the second frequency of the second stimulus signal and the first frequency of the first stimulus signal.

26. The electronic device of claim 25, wherein
the first frequency is a default frequency or a selected frequency associated with a desired SNR, the second frequency is a frequency by adding the first frequency with an increment frequency, and the third frequency is a frequency by subtracting the first frequency with a decrement frequency.

27. The electronic device of claim 25, wherein the processing circuit is configured to:
specify the first condition to the driving circuit for a first period of time;
receive the first feedback signal from the driving circuit;
determine a first magnitude of the first feedback signal;
specify the second condition to the driving circuit for a second period of time;
receive the second feedback signal from the driving circuit;
determine a second magnitude of the second feedback signal;
specify the third condition to the driving circuit for a third period of time;
receive the third feedback signal from the driving circuit;
determine a third magnitude of the third feedback signal.

28. The electronic device of claim 27, wherein the processing circuit is also configured to:
determine a highest magnitude among the first magnitude, the second magnitude, and the third magnitude;
wherein in response to a determination by the processing circuit, that the first magnitude is the highest magnitude, the processing circuit is caused to:
specify the first condition to the driving circuit for the third period of time;
wherein in response to a determination by the processing circuit, that the second magnitude is the highest magnitude, the processing circuit is caused to:
specify the second condition to the driving circuit for the fourth period of time;
wherein in response to a determination by the processing circuit, that the third magnitude is the highest magnitude, the processing circuit is caused to:
specify the third condition to the driving circuit for a fifth period of time.

29. The electronic device of claim 21, wherein the first magnitude of the first feedback signal is selected from a group consisting of: a signal to noise ratio, and a maximum magnitude.

30. The electronic device of claim 21, wherein the first period of time and the second period of time are substantially similar in duration; and
wherein the first period of time is smaller than the third period of time.

31. A method for an electronic device, the electronic device having a first electrode contacting a first portion of a user and a second electrode contacting a second portion of the user, comprising:
determining, with the driving circuit, a first condition indicating that a first stimulus signal is to be output between the first electrode and the second electrode;
wherein in response to determining, by the driving circuit, the first condition, the method further comprising:
determining, with the driving circuit, a first primary frequency for the first stimulus signal;
initiating output, with the driving circuit, of the first stimulus signal between the first electrode and the second electrode; and
determining, with the driving circuit, a first feedback signal from the first electrode and the second electrode in response to the output of the first stimulus signal between the first electrode and the second electrode;
determining, with the driving circuit, a second condition indicating that a second stimulus signal is to be output between the first electrode and the second electrode;
wherein in response to determining, by the driving circuit, the second condition, the method further comprising:
determining, with the driving circuit, a second primary frequency for the second stimulus signal;
initiating output, with the driving circuit, if the second stimulus signal between the first electrode and the second electrode; and
determining, with the driving circuit, a second feedback signal from the first electrode and the second electrode in response to the output of the second stimulus signal between the first electrode and the second electrode;
wherein the first frequency of the first stimulus signal is different from the second frequency of the second stimulus signal.

32. The method of claim 31, further comprising:
in response to a determination, by the driving circuit, of a third condition, determine, with the driving circuit, a third primary frequency for a third stimulus signal;
initiate, with the driving circuit, output of the third stimulus signal between the first electrode and the second electrode; and
determine, with the driving circuit, a third feedback signal from the first electrode and the second electrode in response to the output of the third stimulus signal between the first electrode and the second electrode;
wherein the third frequency of the third stimulus signal is different from the second frequency of the second stimulus signal and the first frequency of the first stimulus signal.

33. The method of claim 32, further comprising:
specifying, with a processing circuit, a first condition to a driving circuit for a first period of time;
receiving, with the processing circuit, the first feedback signal from the driving circuit;
determining, with the processing circuit, a first magnitude of the first feedback signal;
specifying, with the processing circuit, a second condition to the driving circuit for a second period of time;
receiving, with the processing circuit, the second feedback signal from the driving circuit;
determining, with the processing circuit, a second magnitude of the second feedback signal;
determine, with the processing circuit, whether the first magnitude exceeds the second magnitude;
in response to a determination by the processing circuit, that the first magnitude exceeds the second magnitude, specify, with the processing circuit, the first condition to the driving circuit for a third period of time; or
determine, with the processing circuit, whether the second magnitude exceeds the first magnitude;
in response to a determination by the processing circuit, that the second magnitude exceeds the first magnitude, specify, with the processing circuit, the second condition to the driving circuit for a fourth period of time;
specify, with the processing circuit, the first condition to the driving circuit for a first period of time;
receive, with the processing circuit, the first feedback signal from the driving circuit;
determine, with the processing circuit, a first magnitude of the first feedback signal;
specify, with the processing circuit, the second condition to the driving circuit for a second period of time;
receive, with the processing circuit, the second feedback signal from the driving circuit;
determine, with the processing circuit, a second magnitude of the second feedback signal;
specify, with the processing circuit, the third condition to the driving circuit for a third period of time;
receive, with the processing circuit, the third feedback signal from the driving circuit;
determine, with the processing circuit, a third magnitude of the third feedback signal;
determine, with the processing circuit, a highest magnitude among the first magnitude, the second magnitude, and the third magnitude;
in response to a determination by the processing circuit, that the first magnitude is the highest magnitude, specify, with the processing circuit, the first condition to the driving circuit for the third period of time;
in response to a determination by the processing circuit, that the second magnitude is the highest magnitude, specify, with the processing circuit, the second condition to the driving circuit for the fourth period of time;
in response to a determination by the processing circuit, that the third magnitude is the highest magnitude, specify, with the processing circuit, the third condition to the driving circuit for a fifth period of time.

34. The method of claim 31, wherein the first magnitude of the first feedback signal is selected from a group consisting of: a signal to noise ratio, and a maximum magnitude.

35. The method of claim 31, wherein the first period of time and the second period of time are substantially similar in duration; and
wherein the first period of time is smaller than the third period of time.

36. A tuning circuit, comprising:
a first electrode configured to be disposed against a first location on a test surface;
a second electrode configured to be disposed against a second location on the test surface; and
a processing module electrically coupled to the first electrode and the second electrode,
wherein the processing module is configured to provide a first AC voltage signal characterized by a first frequency across the first electrode and the second electrode;
wherein the processing module is configured to measure a first current flowing between the first electrode and the second electrode in response to the first AC voltage signal;
wherein the processing module is configured to determine a first responsive signal in response to the first current and to the first AC voltage signal;
wherein the processing module is configured to provide a second AC voltage signal characterized by a second frequency across the first electrode and the second electrode;
wherein the second frequency is different from the first frequency;
wherein the processing module is configured to measure a second current flowing between the first electrode and the second electrode in response to the second AC voltage signal;
wherein the processing module is configured to determine a second responsive signal in response to the second current and to the second AC voltage signal; thereafter
wherein the processing module is configured to determine if the first responsive signal is greater than the second responsive signal;
wherein in response to determining if the first responsive signal is greater than the second responsive signal;
the processing module is configured to provide a third AC voltage signal characterized by the first frequency across the first electrode and the second electrode;
wherein the processing module is configured to measure a third current flowing between the first electrode and the second electrode in response to the third AC voltage signal; and
wherein the processing module is configured to determine a first electrodermal activity factor in response to the third current and to the third AC voltage signal.

37. The tuning circuit of claim 36, wherein a first magnitude of the first AC voltage signal, a second magnitude of the second AC voltage signal, and a third magnitude of the third AC voltage signal are substantially similar.

38. The tuning circuit of claim 36, wherein in response to determining if the first responsive signal is not greater than the second responsive signal:
the processing module is configured to provide a fourth AC voltage signal characterized by the second frequency across the first electrode and the second electrode;
wherein the processing module is configured to measure a fourth current flowing between the first electrode and the second electrode in response to the fourth AC voltage signal; and
wherein the processing module is configured to determine a second electrodermal activity factor in response to the fourth current and to the fourth AC voltage signal.

39. The tuning circuit of claim 36, wherein the tuning circuit configured in an electronic device that is applied in electrodermal activity (EDA) data collection, the test surface is selected from a group consisting of: a wrist, a finger, fingers, a palm, a foot and a portion of a body.

40. The tuning circuit of claim 36,
wherein the processing module is configured to provide the first AC voltage signal characterized by the first frequency across the first electrode and the second electrode in response to an event; and
wherein the event is selected from a group consisting of: elapse of an amount of time, when a magnitude of the third current flowing between the first electrode and the second electrode is less than a predetermined magnitude, upon initialization of the tuning circuit, at a predetermined time of day.
